SEQUENCE LISTING (5 sequences)

SEQ ID NO: 1 - DNA, 12 bp, Artificial
  Synthetic DNA
tgcgtcgatctc

SEQ ID NO: 2 - DNA, 12 bp, Artificial
  Synthetic DNA
aaaccctgacct

SEQ ID NO: 3 - DNA, 512 bp, Artificial
  Synthetic DNA (5′-0-vinylated DNA)
tgcgtcgatctctgggaaccgtacctgatctctgcgtgaaaggaatacgatagtgagcgagcacgtcaataagtacacggagtacgaggcacgtaccaaggcgatcgaaaccttgctgtacgagcgagggctcatcacgcccgccgcggtcgaccgagtcgtttcgtactacgagaacgagatcggcccgatgggcggtgccaaggtcgtggccaagtcctgggtggaccctgagtaccgcaagtggctcgaagaggacgcgacggccgcgatggcgtcattgggctatgccggtgagcaggcacaccaaatttcggcggtcttcaacgactcccaaacgcatcacgtggtggtgtgcactctgtgttcgtgctatccgtggccggtgcttggtctcccgcccgcctggtacaagagcatggagtaccggtcccgagtggtagcggaccctcgtggagtgctcaagcgcgatttcggtttcgacatccccgatgaggtggaggtcagggttt

SEQ ID NO: 4 - DNA, 512 bp, Artificial; identical to SEQ ID NO: 3
  Synthetic DNA

SEQ ID NO: 5 - DNA, 512 bp, Artificial; identical to SEQ ID NO: 3
  Synthetic DNA

(19) United States
(12) Patent Application Publication
(10) Pub. No.: US 2006/0252086 A1
(43) Pub. Date: Nov. 9, 2006

(54) Process for producing vinylated nucleic acid

(75) Inventors: Toshitaka Uragaki, Tokyo (JP); Fumiaki Watanabe, Kanagawa (JP); Chiaki Nagahama, Kanagawa (JP); Fujio Yu, Kanagawa (JP); Katsuaki Kikuchi, Kanagawa (JP); Akira Yoshioka, Kanagawa (JP)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignee: Mitsubishi Rayon Co., Limited, Tokyo (JP)

(21) Appl. No.: 11/438,463

(22) Filed: May 23, 2006

Related U.S. Application Data
(63) Continuation of application No. 10/470,731, filed on Aug. 8, 2003.

(30) Foreign Application Priority Data
Feb. 8, 2001 (JP) 2001-032030

(51) Int. Cl. C12Q 1/68; C12P 19/34; C07H 21/04

(52) U.S. Cl. 435/6; 536/25.3; 435/91.2

(57) ABSTRACT

This invention provides a process for producing a vinylated nucleic acid in an efficient and cost-effective manner through the implementation of vinylation of an amino-containing nucleic acid with a vinylating agent. This invention also provides a process for producing a vinylated nucleic acid in an efficient and cost-effective manner by a method in which PCR is carried out in the presence of an amino-modified nucleotide and dNTP and a vinyl group is introduced into the resulting PCR amplification product or by a method in which PCR is carried out in the presence of a vinyl-modified nucleotide and cNTP.

### CONTINUING APPLICATION DATA

The present application is a Continuation of U.S. application Ser. No. 10/470,731, filed on Aug. 8, 2003, pending, which is the National Stage of PCT/JP02/01108, filed Feb. 8, 2002.

### TECHNICAL FIELD

The present invention relates to a process for producing a vinylated nucleic acid having a vinyl group. The vinylated nucleic acid is immobilized in a substrate, etc. to be used as a probe for detecting gene expression, gene variation, or the like.

### BACKGROUND ART

In recent years, DNA microarrays, rnicroelectrophoresis apparatuses, and the like have been developed as devices for analyzing genomes or gene information.

For example, some of the present inventors have developed a DNA microarray utilizing gel and filed for patents (see JP Patent Publication (Kokai) Nos. 2000-270877 A, 2000-270878 A, and 2000-270879 A). This DNA microarray is obtained by preparing an arranged body of fibers holding a nucleic acid-immobilized gel thereon and cutting the arranged body in a direction that intersects the fiber axis thereof.

An example of a known process for immobilizing a nucleic acid in gel involves a nucleic acid having a vinylated terminus, which is prepared using acrylamide phosphoramidite (Acrydite™) as a vinylating agent. This nucleic acid is copolymerized with an acrylamide monomer, thereby immobilizing the nucleic acid in polyacrylamide (see Nucleic Acid Res., 27. 2649 (1999) and WO 98/39351). Since the introduction of a vinyl group into the nucleic acid is unstable, however, a vinyl group cannot be efficiently introduced (BioTechniques 27:592-606 (1999)). Since the phosphoramidite reagent is expensive, this process is not economical.

### DISCLOSURE OF THE INVENTION

An object of the present invention is to provide a process for producing a vinylated nucleic acid in an efficient and cost-effective manner.

The present inventors have conducted concentrated studies in order to attain the above object. As a result, they have found that a vinylated nucleic acid can be produced in an efficient and cost-effective manner by allowing an amino-containing nucleic acid to react with a vinylating agent, and if necessary, performing vinylation in the presence of a basic compound. This has led to the completion of the present invention.

The present inventors have also found that a vinylated nucleic acid can be produced in an efficient and cost-effective manner by a process in which PCR is carried out in the presence of an amino-modified nucleotide and a vinyl group is introduced into the resulting PCR amplification product or by PCR in the presence of a vinyl-containing nucleotide. This has also led to the completion of the present invention.

More specifically, the present invention relates to: (1) a process for producing a vinylated nucleic acid, wherein an amino-containing nucleic acid is subjected to vinylation with a vinylating agent; (2) the process for producing a vinylated nucleic acid according to (1), wherein the amino-containing nucleic acid is obtained by PCR; (3) the process for producing a vinylated nucleic acid according to (1), wherein the amino-containing nucleic acid is obtained by PCR in the presence of an amino-modified nucleotide and dNTP; (4) the process for producing a vinylated nucleic acid according to any one of (1) to (3), wherein the vinylating agent is at least one member selected from the group consisting of acrylic anhydride, methacrylic anhydride, N-acryloyloxy succinimide, and N-methacryloyloxy succinimide; (5) the process for producing a vinylated nucleic acid according to any one of (1) to (4), wherein the vinylation is carried out in the presence of a basic compound; (6) a process for producing a vinylated nucleic acid, wherein PCR is carried out using, as a primer, the vinylated nucleic acid obtained by the process according to any one of (1) to (5), thereby obtaining a vinylated nucleic acid as a PCR amplification product; (7) a process for producing a vinylated nucleic acid, wherein PCR is carried out in the presence of a vinyl-modified nucleotide and dNTP, thereby obtaining a vinylated nucleic acid as a PCR amplification product; (8) the process for producing a vinylated nucleic acid according to (7), wherein the vinyl-modified nucleotide is obtained by vinylating an amino-modified nucleotide with a vinylating agent; (9) the process for producing a vinylated nucleic acid according to (8), wherein the vinylating agent is at least one member selected from the group consisting of acrylic anhydride, methacrylic anhydride, N-acryloyloxy succinimide, and N-methacryloyloxy succinimide; and (10) the process for producing a vinylated nucleic acid according to (8) or (9), wherein the vinylation is carried out in the presence of a basic compound.

The present invention is hereafter described in detail.

The “vinylated nucleic acid” that is produced in the present invention refers to a nucleic acid comprising one or several vinyl-containing nucleotides incorporated in its nucleic acid sequence. The phrase “in its nucleic acid sequence” refers to the inside and/or terminus of the nucleic acid sequence. “PCR” refers to the polymerase chain reaction.

The vinylated nucleic acid of the present invention is produced by any of the following processes:

(A) an amino-containing nucleic acid is allowed to react with a vinylating agent;

(B) PCR is carried out in the presence of an amino-modified nucleotide and dNTP, and the PCR amplification product is then allowed to react with a vinylating agent; or

(C) PCR is carried out in the presence of a vinyl-modified nucleotide and dNTP.

In the process described in (A), an “amino-containing nucleic acid” can be synthesized as follows. For example, a nucleic acid comprising a suitable number of bases is synthesized using an amidite reagent in an automatic DNA synthesizer, and at the final phase, an aminating reagent such as Aminolink™ (PE Biosystems) is allowed to react thereon, followed by deprotection. In this case, an amino group is introduced at the terminus of the nucleic acid. This nucleic acid comprises approximately 100 bases.

When intending to obtain a long-chain amino-containing nucleic acid comprising 100 or more bases, preparation thereof by PCR is effective. At the outset, a nucleic acid of a suitable length having an amino group at its terminus is prepared using an automatic DNA synthesizer or the like, and the resultant is used as a primer at the time of PCR. Thus, a long-chain nucleic acid having an amino group at its terminus can be synthesized. PCR can be carried out in accordance with a conventional process.

The aforementioned nucleic acid having an amino group at its terminus is allowed to react with a vinylating agent, thereby obtaining a nucleic acid comprising vinylation introduced at its terminus (this may be referred to as a “nucleic acid having a vinylated terminus”).

A vinylating agent is selected in consideration of reactivity with a polar solvent, which is a good solvent for an amino-containing nucleic acid, for example, dimethyl sulfoxide (DMSO), and reactivity with an amino group in a nucleic acid base. A preferable vinylating agent is a compound containing an acryl, methacryl, or other group. Specific examples thereof are acrylic anhydride, methacrylic anhydride, acrylic acid N-hydroxysuccinimide ester (N-acryloyloxy succinimide), and methacrylic acid N-hydroxysuccinimide ester (N-methacryloyloxy succinimide).

The amount of the vinylating agent to be used in the reaction is determined in consideration of the reaction yield. From an economic point of view, an equimolar to 50-fold molar amount of the vinylating agent is preferably used relative to a nucleic acid having an amino group at its terminus.

The reaction temperature in vinylation is arbitrarily determined in consideration of the reaction speed, reaction yield, or the like. It is preferably between 10° C. and 30° C.

The use of a basic compound as a catalyst in the vinylation of an amino-containing nucleic acid can significantly improve the reaction speed, reaction yield, or the like of the vinylation.

Examples of a basic compound include: alkaline (earth) metals such as sodium, potassium, or calcium; alkaline (earth) metal hydroxides such as sodium hydroxide, potassium hydroxide, or calcium hydroxide; alkaline (earth) metal carbonate compounds such as sodium carbonate, sodium bicarbonate, potassium carbonate, or potassium bicarbonate; alkaline (earth) metal alkoxy compounds such as sodium methylate or magnesium methylate; alkaline (earth) metal hydrides such as sodium hydride or calcium hydride; and organic tertiary amines such as triethylamine or diazabicycloundecene. These may be used in combinations. Preferable examples include inexpensive sodium carbonate, sodium bicarbonate, sodium hydroxide, and potassium hydroxide. The amount used is preferably an equimolar to 100-fold molar amount relative to a nucleic acid having an amino group at its terminus.

In the process as described in (B), the “amino-modified nucleotide” refers to a nucleotide having an amino group. An example of an amino group is a aliphatic amino group, and a specific example thereof that can be used is a compound such as 5-(3-aminoallyl)-2′-deoxyuridine-5′-triphosphate.

Implementation of PCR in the presence of the amino-modified nucleotide and dNTP (a mixture including DATP, dGTP, dCTP, and dTTP) can yield a nucleic acid having an amino group therein as a PCR amplification product.

Also, the use of, for example, the amino-containing nucleic acid synthesized by the process described in (A) for both or one of a pair of primers to be used in PCR can yield a nucleic acid having an amino group inside and at the terminus of its nucleic acid sequence.

As mentioned above, the resulting PCR amplification product becomes a nucleic acid comprising a vinyl group introduced inside and/or at the terminus of its nucleic acid sequence upon reaction with a vinylating agent.

The number of vinyl groups in the nucleic acid sequence can be arbitrarily determined depending on the amount of amino-modified nucleotides that are added at the time of PCR. The amount of the amino-modified nucleotides that are added at the time of PCR is preferably small from an economic point of view, and an amount of 1.0 to 10.0% by mass relative to dNTP is particularly preferable.

In the process as described in (C), the “vinyl-modified nucleotide” can refer to, for example, an acrylated compound of 5-(3-aminoallyl)-2′-deoxyuridine-5′-triphosphate and a methacrylated compound of 5-(3-aminoallyl)-2′-deoxyuridine-5′-triphosphate. A vinyl-modified nucleotide can be obtained by allowing the amino-modified nucleotide to react with the aforementioned vinylating agent.

Implementation of PCR in the presence of the vinyl-modified nucleotide and dNTP (a mixture including DATP, dGTP, dCTP, and dTTP) can yield a nucleic acid having an amino group therein as a PCR amplification product.

Also, the use of, for example, the vinylated nucleic acid synthesized by the process described in (A) for both or one of a pair of primers to be used in PCR can yield an nucleic acid comprising a vinyl group introduced inside and at the terminus of its nucleic acid sequence.

As with the case of the process as described in (B) above, the number of vinyl groups in the nucleic acid can be arbitrarily determined depending on the amount of vinyl-containing nucleotides that are added at the time of PCR. The amount of the vinyl-containing nucleotides that are added at the time of PCR is preferably small from an economic point of view, and an amount of 1.0 to 10.0% by mass relative to DNTP is particularly preferable.

In the present invention, the introduction of a vinyl group in the nucleic acid can be confirmed by preparing a copolymer through the reaction between a vinylated nucleic acid and a polymerizable monomer such as acrylamide, and subjecting the copolymer to electrophoresis.

The vinylated nucleic acid is copolymerized with a monomer such as acrylamide, and thus it does not migrate because of the electrophoresis.

The nucleic acid that is immobilized in a copolymer with a polymerizable monomer such as acrylamide can be used as a probe for detecting gene expression, gene variation, or the like.

### BRIEF DESCRIPTION OF THE DRAWING

FIG. 1 shows an electrophoresis gel and a process for confirming a vinylated nucleic acid using the same, wherein reference numeral 1 indicates a nucleic acid-immobilized gel, reference numeral 2 indicates an electrophoresis gel, and reference numerals 11 to 14 indicate a nucleic acid-immobilized gel or a well to which a nucleic acid solution is added.

### BEST MODES FOR CARRYING OUT THE INVENTION

The present invention is hereafter described in more detail with reference to the examples, although the technical scope of the present invention is not limited to these examples.

### EXAMPLE 1

The nucleic acid of atgc was synthesized using an amidite reagent in an automatic DNA synthesizer, and at the final phase, Aminolink™ (PE Biosystems) was allowed to react thereon, followed by deprotection. Thus, 5′-O-aminohexyl-atgc was synthesized.

An aqueous solution (10 μl) of 1 mM 5′-O-aminohexyl-atgc was used as a nucleic acid having an amino group at its terminus, and 5 μl of solution of 80 mM methacrylic anhydride (dissolved in DMSO) was used as a vinylating agent. An aqueous solution (5 μl) of 100 mM sodium carbonate was mixed with the aforementioned, and the mixture was subjected to vinylation at room temperature for 1 hour. The molar ratio of the nucleic acid having an amino group at its terminus to methacrylic anhydride was 1:40.

The reaction yield was assayed by liquid chromatography under the following conditions for analysis. As a result, the reaction yield was 100%.
(Conditions for liquid chromatography analysis)
Column: Capcell Pak C18 SG300 (4.6 mm i.d.×250 mm, 5 μm)
Mobile phase: A: 5 mM triethylamine-acetic acid (pH 7.5)

B: acetonitrile 0→20% (40 min)
Detection: UV 260 nm
Flow rate: 1.0 m/min

### EXAMPLES 2 TO 5

The same procedure as in Example 1 was performed except that the concentration of a solution of methacrylic anhydride was changed to the values as shown in Table 1. The results are shown in Table 1.

TABLE 1

| | Methacrylic anhydride (mM) | Reaction yield (%) |
|---|---|---|
| Example 2 | 20 | 100 |
| Example 3 | 10 | 99.1 |
| Example 4 | 5 | 97.4 |
| Example 5 | 2.5 | 26.5 |

### COMPARATIVE EXAMPLE 1

The same procedure as in Example 1 was performed except that an aqueous solution of 100 mM methacrylic acid was used instead of methacrylic anhydride. After the completion of the reaction, the reaction yield was assayed by liquid chromatography. As a result, the reaction yield was 0%.

### EXAMPLES 6 TO 10

The same procedures as in examples 1 to 5 were performed except that N-acryloyloxy succinimide was used instead of methacrylic anhydride, and the reaction yield was determined.

TABLE 2

| | N-acryloyloxy succinimide (mM) | Reaction yield (%) |
|---|---|---|
| Example 6 | 80 | 100 |
| Example 7 | 20 | 99.5 |
| Example 8 | 10 | 90.8 |
| Example 9 | 5 | 90.8 |
| Example 10 | 2.5 | 30.1 |

### EXAMPLE 11

The nucleic acid of tgcgtcgatctc (SEQ ID NO: 1) was synthesized using an amidite reagent in an automatic DNA synthesizer, and at the final phase, Aminolink™ (PE Biosystems) was allowed to react thereon, followed by deprotection. Thus, 5′-O-aminohexyl-tgcgtcgatctc was synthesized.

An aqueous solution (10 μl) of 0.5 mM 5′-O-aminohexyl-tgcgtcgatctc was used as a nucleic acid having an amino group at its terminus, and 5 μl of solution of 20 mM methacrylic anhydride (dissolved in DMSO) was used as a vinylating agent. These solutions were mixed with 5 μl of aqueous solution of 100 mM sodium carbonate, and the mixture was subjected to reaction at room temperature for 1 hour.

The reaction yield was assayed by liquid chromatography under the same conditions as in Example 1. As a result, the reaction yield was 100%.

### EXAMPLE 12

This example relates to a process for producing a nucleic acid having a vinylated terminus by PCR using a nucleic acid having a vinylated terminus.

(1) Preparation of a Template (Chromosome)

The Rhodococcus rhodochrous J1 strain was cultured in 100 ml of nutrient medium (15 g of glucose, 1 g of yeast extract, 10 g of sodium glutamate, 0.5g of KH₂PO₄, 0.5g of K₂HPO₄, and 0.5 g/L of MgSO₄.7H₂O, pH 7.2) at 30° C. for 3 days and then collected. A chromosome was prepared from this strain and used as a template for PCR. The Rhodococcus rhodochrous J1 strain was deposited at the International Patent Organism Depositary of the National Institute of Advanced Industrial Science and Technology (Tsukuba Central 6, 1-1-1 Higashi, Tsukuba, Ibaraki, Japan) as of Sep. 18, 1987 under the accession number FERM BP-1478.

(2) PCR

PCR was carried out using the nucleic acid having a vinylated terminus prepared in Example 11 and aaaccctgacct (SEQ ID NO: 2) as primers and the chromosome prepared in (1) as a template. The synthesis of aaaccctgacct (SEQ ID NO: 2) was requested to Amersham Pharmacia.

PCR was carried out using TaKaRa PCR Thermal Cycler PERSONAL in accordance with the instructions of Ex-Taq (Takara Shuzo Co., Ltd.). The reaction was carried out in 100 μl for 30 PCR cycles of 93° C. for 30 seconds, 55° C. for 30 seconds, and 72° C. for 1 minute. In this 512-base-long nucleic acid having the vinylated 5′-terminus (SEQ ID NO: 3) was amplified.

| [SEQ ID NO: 3] | |
|---|---|
| tgcgtcgatc tctgggaacc gtacctgatc tctgcgtgaa aggaatacga tagtgagcga | 60 |
| gcacgtcaat aagtacacgg agtacgaggc acgtaccaag gcgatcgaaa ccttgctgta | 120 |
| cgagcgaggg ctcatcacgc ccgccgcggt cgaccgagtc gtttcgtact acgagaacga | 180 |
| gatcggcccg atgggcggtg ccaaggtcgt ggccaagtcc tgggtggacc ctgagtaccg | 240 |
| caagtggctc gaagaggacg cgacggccgc gatggcgtca ttgggctatg ccggtgagca | 300 |
| ggcacaccaa atttcggcgg tcttcaacga ctcccaaacg catcacgtgg tggtgtgcac | 360 |
| tctgtgttcg tgctatccgt ggccggtgct tggtctcccg cccgcctggt acaagagcat | 420 |
| ggagtaccgg tcccgagtgg tagcggaccc tcgtggagtg ctcaagcgcg atttcggttt | 480 |
| cgacatcccc gatgaggtgg aggtcagggt tt | 512 |

### EXAMPLE 13

This example relates to the immobilization of a nucleic acid having a vinylated terminus in an acrylamide gel and evaluation thereof.

An aqueous solution of the gel precursor as shown in Table 3 was prepared, and allowed to stand at room temperature for 2 hours to produce a nucleic acid-immobilized acrylamide gel. The resulting nucleic acid-immobilized gel 1 (20 mg) was cleaved out. A nucleic acid (5 nmol) was present in 20 mg of the nucleic acid-immobilized gel 1.

TABLE 3

| | | |
|---|---|---|
| Acrylamide | 4.8% | by mass |
| Methylenebisacrylamide | 0.2% | by mass |
| APS (ammonium persulfate) | 0.1% | by mass |
| TMED ((N,N,N′,N′-tetramethylethylenediamine)) | 0.1% | by mass |
| Vinylated nucleic acid (prepared in Example 11) | 250 | μM |

Subsequently, electrophoresis gel 2 (polymer concentration: 5%, (acrylamide/methylenebisacrylamide=95/5 (wt/wt)) containing 4 wells (volume: approximately 50 μl) was prepared.

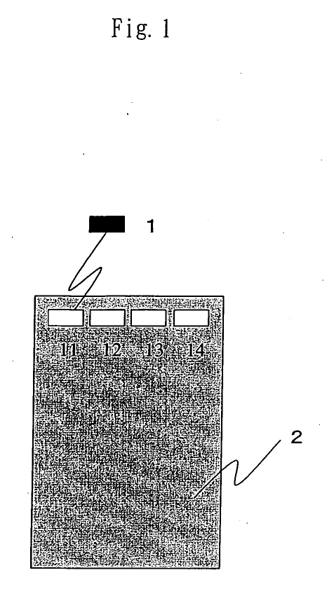

The nucleic acid-immobilized gel 1 that was cleaved out above was added to well 11. Solutions of nucleic acid having a vinylated terminus (described in Example 11) were added to wells 12 to 14 in amounts of 5 nmol, 2.5 nmol, and 0.5 nmol in that order (see FIG. 1).

Subsequently, a submarine electrophoresis apparatus (AE-61 10, Atto) was used to perform electrophoresis at 50 V for 15 minutes.

The acrylamide gel was stained with ethidium bromide, and the fluorescence intensity of the band of the nucleic acid, which had migrated because of the electrophoresis, was visually inspected. The fluorescence intensity of the band of the nucleic acid that had migrated from the well 11 (i.e., a vinylated nucleic acid that was not copolymerized) was found to be lower than that of the well 14.

Accordingly, the ratio of the nucleic acid having a vinylated terminus to be immobilized in an acrylamide gel was confirmed to be 90% or higher.

### EXAMPLE 14

This example relates to the immobilization of a long-chain nucleic acid having a vinylated terminus (SEQ ID NO: 3) in an acrylamide gel and evaluation thereof An electrophoresis gel was prepared in the same manner as in Example 13 except that only one well was used instead of four wells.

A solution of the gel precursor containing a long-chain nucleic acid having a vinylated terminus (SEQ ID NO: 3) having the composition as shown in Table 4 was added to the well portion in the gel, and allowed to stand at room temperature for 2 hours.

TABLE 4

| | |
|---|---|
| Monomer solution (acrylamide/methylenebisacrylamide = 95/5) | 10 μl |
| (25% by mass) | |
| Pure water | 20 μl |
| 1% APS (ammonium persulfate) | 5 μl |
| 10% TMED ((N,N,N′,N′-tetramethylethylenediamine)) | 5 μl |
| Vinylated nucleic acid (SEQ ID NO: 3) | 10 μl |

The resulting gel was subjected to electrophoresis at 50 V for 1 hour using a vertical electrophoresis apparatus, followed by staining with ethidium bromide.

The fluorescence intensity of the well portion in an acrylamide gel to which the sample was to be added (a long chain nucleic acid having a vinylated terminus that was immobilized in gel) was compared to that of the band of the nucleic acid that had migrated because of the electrophoresis (a long chain nucleic acid having a vinylated terminus that was not immobilized in gel). As a result, the fluorescence intensity of the well portion was approximately ten times as high as that of the band based on visual inspection.

PCR was carried out in the same manner as in Example 12 (2) except that a nucleic acid having an amino group at its terminus (5′-O-aminohexyl-tgcgtcgatctc, see Example 11) was used instead of the nucleic acid having a vinylated terminus to obtain a PCR amplification product. A similar experiment was conducted using the resulting PCR amplification product instead of the vinylated nucleic acid (SEQ ID NO: 3) shown in Table 4. As a result, the well portion was not stained with ethidium bromide, although only the band, which had migrated because of the electrophoresis, was intensely stained.

Accordingly, PCR was carried out using a nucleic acid having a vinylated terminus as a primer. The resulting long chain nucleic acid having a vinylated terminus (SEQ ID NO: 3) was also found to be efficiently immobilized in an acrylamide gel.

### EXAMPLE 15

This example relates to a process for producing a vinylated nucleic acid by PCR in the presence of an amino-modified nucleotide and dNTP.

(1) PCR

PCR was carried out using the template prepared in Example 12 (1) and tgcgtcgatctc (SEQ ID NO: 1) and aaaccctgacct (SEQ ID NO: 2) as primers. The synthesis of tgcgtcgatctc (SEQ ID NO: 1) and aaaccctgacct (SEQ ID NO: 2) was requested to Amersham Pharmacia.

PCR was carried out in the same manner as in Example 12 except that 5-(3-aminoallyl)-2′-deoxyuridine-5′-triphosphate was added as a nucleotide in an amount of 0.05 relative to 1 DNTP. As a result, a 512-base-long nucleic acid (SEQ ID NO: 4) comprising amino-modified nucleotides in its sequence was amplified.

| [SEQ ID NO: 4] | |
|---|---|
| tgcgtcgatc tctgggaacc gtacctgatc tctgcgtgaa aggaatacga tagtgagcga | 60 |
| gcacgtcaat aagtacacgg agtacgaggc acgtaccaag gcgatcgaaa ccttgctgta | 120 |
| cgagcgaggg ctcatcacgc ccgccgcggt cgaccgagtc gtttcgtact acgagaacga | 180 |
| gatcggcccg atgggcggtg ccaaggtcgt ggccaagtcc tgggtggacc ctgagtaccg | 240 |
| caagtggctc gaagaggacg cgacggccgc gatggcgtca ttgggctatg ccggtgagca | 300 |
| ggcacaccaa atttcggcgg tcttcaacga ctcccaaacg catcacgtgg tggtgtgcac | 360 |
| tctgtgttcg tgctatccgt ggccggtgct tggtctcccg cccgcctggt acaagagcat | 420 |
| ggagtaccgg tcccgagtgg tagcggaccc tcgtggagtg ctcaagcgcg atttcggttt | 480 |
| cgacatcccc gatgaggtgg aggtcagggt tt | 512 |

(2) Introduction of a Vinyl Group

The nucleic acid (SEQ ID NO: 4) (10 μl, 1 nmol/ml) obtained in (1) was used as an amino-containing nucleic acid, and 5 μl of solution of 50 mM methacrylic anhydride (dissolved in DMSO) was used as a vinylating agent. An aqueous solution (5 μl) of 100 mM Na₂CO₃—NaHCO₃ was mixed with the aforementioned, and the mixture was allowed to react at room temperature for 2 hours.

(3) Immobilization of Vinylated Nucleic Acid in Acrylamide Gel and Evaluation Thereof

The same procedure as in Example 14 was performed except that the vinylated nucleic acid obtained in (2) was used.

The fluorescence intensity of the well portion in an acrylamide gel to which the sample was to be added (a vinylated nucleic acid that was immobilized in gel) was compared to that of the band of the nucleic acid that had migrated because of the electrophoresis (a vinylated nucleic acid that was not immobilized in gel). As a result, the fluorescence intensity of the well portion was approximately ten times as high as that of the band based on visual inspection.

### EXAMPLE 16

This example relates to a process for producing a vinylated nucleic acid by PCR in the presence of a vinyl-modified nucleotide and dNTP.

(1) Synthesis of Vinyl-Modified Nucleotide

5-(3-Aminoallyl)-2′-deoxyuridine-5′-triphosphate (50 mM, 10 μl, dissolved in DMSO) was used as an amino-modified nucleotide, and 5 μl of 50 mM methacrylic anhydride (dissolved in DMSO) was used as a vinylating agent. 5 μl of 100 mM Na₂CO₃—NaHCO₃ was mixed with the aforementioned, and the mixture was allowed to react at room temperature for 2 hours.

(2) PCR

PCR was carried out in the same manner as in Example 15 except that the nucleotide synthesized in (1) was added in an amount of 0.05 relative to 1 DNTP. As a result, a 512-base-long nucleic acid (SEQ ID NO: 5) comprising vinyl-modified nucleotides in its sequence was amplified.

| [SEQ ID NO: 5] | |
|---|---|
| tgcgtcgatc tctgggaacc gtacctgatc tctgcgtgaa aggaatacga tagtgagcga | 60 |
| gcacgtcaat aagtacacgg agtacgaggc acgtaccaag gcgatcgaaa ccttgctgta | 120 |
| cgagcgaggg ctcatcacgc ccgccgcggt cgaccgagtc gtttcgtact acgagaacga | 180 |
| gatcggcccg atgggcggtg ccaaggtcgt ggccaagtcc tgggtggacc ctgagtaccg | 240 |
| caagtggctc gaagaggacg cgacggccgc gatggcgtca ttgggctatg ccggtgagca | 300 |
| ggcacaccaa atttcggcgg tcttcaacga ctcccaaacg catcacgtgg tggtgtgcac | 360 |
| tctgtgttcg tgctatccgt ggccggtgct tggtctcccg cccgcctggt acaagagcat | 420 |
| ggagtaccgg tcccgagtgg tagcggaccc tcgtggagtg ctcaagcgcg atttcggttt | 480 |
| cgacatcccc gatgaggtgg aggtcagggt tt | 512 |

(3) Immobilization of Vinylated Nucleic Acid (SEQ ID NO: 5) in an Acrylamide Gel and Evaluation Thereof

The same procedure as in Example 14 was performed except for the use of the vinylated nucleic acid (SEQ ID NO: 5) obtained in (2).

The fluorescence intensity of the well portion in an acrylamide gel to which the sample was to be added (a vinylated nucleic acid that was immobilized in gel) was compared to that of the band of the nucleic acid that had migrated because of the electrophoresis (a vinylated nucleic acid that was not immobilized in gel). As a result, the fluorescence intensity of the well portion was approximately ten times as high as that of the band based on visual inspection.

### INDUSTRIAL APPLICABILITY

In the present invention, an amino-containing nucleic acid is subjected to vinylation with a vinylating agent. Thus, a vinylated nucleic acid can be produced in an efficient and cost-effective manner. Also, a vinylated nucleic acid can be produced in an efficient and cost-effective manner by a process in which PCR is carried out in the presence of an amino-modified nucleotide and dNTP and a vinyl group is introduced into the resulting PCR amplification product or by a process in which PCR is carried out in the presence of a vinyl-modified nucleotide and DNTP.

All publications cited herein are incorporated herein in their entirety. A person skilled in the art would easily understand that various modifications and changes of the present invention are feasible within the technical idea and the scope of the invention as disclosed in the attached claims. The present invention is intended to include such modifications and changes.

1. A process for producing a vinylated nucleic acid, wherein an amino-containing nucleic acid is subjected to vinylation with a vinylating agent.

2. The process for producing a vinylated nucleic acid according to claim 1, wherein the amino-containing nucleic acid is obtained by PCR.

3. The process for producing a vinylated nucleic acid according to claim 1, wherein the amino-containing nucleic acid is obtained by PCR in the presence of an amino-modified nucleotide and dNTP.

4. The process for producing a vinylated nucleic acid according to any one of claims 1 to 3, wherein the vinylating agent is at least one member selected from the group consisting of acrylic anhydride, methacrylic anhydride, N-acryloyloxy succinimide, and N-methacryloyloxy succinimide.

5. The process for producing a vinylated nucleic acid according to any one of claims 1 to 4, wherein the vinylation is carried out in the presence of a basic compound.

6. A process for producing a vinylated nucleic acid, wherein PCR is carried out using, as a primer, the vinylated nucleic acid obtained by the process according to any one of claims 1 to 5, thereby obtaining a vinylated nucleic acid as a PCR amplification product.

7. A process for producing a vinylated nucleic acid, wherein PCR is carried out in the presence of a vinyl-modified nucleotide and dNTP, thereby obtaining a vinylated nucleic acid as a PCR amplification product.

8. The process for producing a vinylated nucleic acid according to claim 7, wherein the vinyl-modified nucleotide is obtained by vinylating an amino-modified nucleotide with a vinylating agent.

9. The process for producing a vinylated nucleic acid according to claim 8, wherein the vinylating agent is at least one member selected from the group consisting of acrylic anhydride, methacrylic anhydride, N-acryloyloxy succinimide, and N-methacryloyloxy succinimide.

10. The process for producing a vinylated nucleic acid according to claim 8 or 9, wherein the vinylation is carried out in the presence of a basic compound.
